(19) United States
(12) Patent Application Publication
(10) Pub. No.: US 2012/0330356 A1
(43) Pub. Date: Dec. 27, 2012

(54) SUTURE CLIP STAPLER FOR SOFT TISSUE CLOSURE

(76) Inventor: Paul H. Rosenberg, Fort Lee, NJ (US)

(21) Appl. No.: 13/513,281

(22) PCT Filed: Dec. 1, 2010
(86) PCT No.: PCT/US10/03066

§ 371 Date: Sep. 7, 2012

Related U.S. Application Data
(60) Provisional application No. 61/265,552, filed on Dec. 1, 2009.

(51) Int. Cl. A61B 17/06

(52) U.S. Cl. 606/232

(57) ABSTRACT

A suture clip is comprised of a length of suture material with a male clip on one side and a female clip on the other side. The male clip may contain ratchets that, when engaged with the female clip, hold it in place and allow the male clip to adjustably lit within the female clip or even through it to “tighten” the suture when the male and female clips are engaged. The suture clips are configured to be used in a suture clip applicator that contains paired and opposing needles configured to hold the male clip on one side and the female clip on the other side and to cause the engagement of male and female clips and form a continuous loop through the suture material to enable the closure of fascia or other body tissues.

### FIELD OF THE INVENTION

The present invention relates to a device, apparatus and method for application of a combination of a suture and a clip for reconnecting body tissues, for example alter surgical procedures or trauma. More specifically, a suture clip is comprised of a suture having a male connector on one end and a female connector on other end. Multiple suture clips may be loaded into a suture clip applicator that enables automatic application of the suture clips to body tissue by actuation of the device.

### BACKGROUND OF THE INVENTION

The present invention provides a method of saving time and ease of use in closing a layer of body tissue, such as ligaments, fascia, flaps, grafts or even sternal wounds, that result from surgery or trauma. Although the invention may be used in many different contexts, use of the invention during body lifts and abdominoplasty will be described solely for illustration purposes.

The fascia is the supportive tissue layers on top of the muscles of the abdomen. This is the layer that is plicated during an abdominoplasty, also known as a “tummy tuck.” A 4 to 10 or more inch width of this fascia is brought in the midline together and closed with non-absorbable sutures so that it acts as an internal corset to tighten the abdomen. This technique is widely used in both cosmetic abdominoplasty and in body procedures commonly performed alter massive weight loss, where excess skin is trimmed and the underlying structures are tightened in recontouring the body

The object of the abdominoplasty, aside from tightening the skin and contouring the flanks, is to sew together the fascia from one side, roughly about midway from one rectus abdominus muscle, to midway on the other side. There are two widely used ways of accomplishing this. One method is to run a continuous nonabsorbable suture from the region of the xyphoid process to the region of the pubis. A second method of fascial plication is to use multiple figure-of-eight sutures. Figure-of-eight sutures are a single pass from one side to the other followed by another pass in such as way that the suture resembles an “8”. In this method, there is a transverse and a vertical component to the closure. It is a tighter fascial closure and there are generally ten to twelve sutures from the zyphoid process to the pubis. Each suture takes up to a minute or so for the surgeon to place. Thus, the process is time consuming and tends to use a large amount of suture, especially if each suture is hand tied, which tends to be expensive. Although more lime consuming and expensive, this method is the preferred way of plicating the fascia. This is because even if one or two sutures comes loose, the majority of the closure is still intact and the remaining sutures will not loosen up as the continuous suture is known to do.

It is with these considerations in mind the present invention is contemplated.

### BRIEF DESCRIPTION OF THE DRAWINGS

The foregoing and other features of the present invention will be more readily apparent from the Following detailed description and drawings of the illustrative embodiments of the invention in which:

FIG. 1 is a perspective view of the suture clip of the present invention;

FIG. 2 is a second perspective view of the suture clip of the present invention;

FIG. 3 is cross sectional view of the engaged clip portions of the present invention;

FIG. 4 is a perspective cross-sectional view of the clip portions of the present invention;

FIG. 5 is an alternative embodiment of the suture clip of the present invention;

FIG. 6 is an alternative embodiment of the suture clip of the present invention wherein the male and female clips are engaged;

FIG. 7 is another alternative embodiment of the suture clip of the present invention;

FIG. 8 is a front view of another embodiment of the suture clip of the present invention with the clips engaged;

FIG. 9 is a perspective view of another embodiment of the present invention with the clip pieces engaged;

FIG. 10 is perspective view of a suture clip applicator of the present invention in the unactuated position;

FIG. 11 is a perspective view of the suture clip applicator of the present invention in a partially actuated position;

FIG. 12 is a perspective view of a suture clip applicator, partially actuated with the top cover removed;

FIG. 13 is the suture clip applicator of the present invention fully actuated with the cover removed;

FIG. 14 is an exploded view of the suture clip applicator;

FIG. 15 is a perspective view of the needle assembly of the present invention;

FIGS. 16A-C is the needle assembly in various positions of actuation;

FIG. 17 is an exploded view of the needle assembly of the present invention;

FIG. 18 shows a top front view of the suture clip applicator of the present invention;

FIG. 19 shows a sectional view oldie needle assembly of the present invention;

FIGS. 20A-C show the actuation of the suture clip applicator when applied to soft tissue;

FIG. 21 shows the application of an alternative embodiment of a suture clip applicator and alternative embodiment of a suture clip;

FIG. 22 shows the alternative embodiment suture clip when engaged;

FIG. 23 shows a cartridge for the suture clip applicator; and

FIG. 24 shows an exploded view of a cartridge of suture clips.

### DETAILED DESCRIPTION

Various embodiments of the present invention will be described in detail with reference to the drawings, where like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

A suture clip is comprised of a length of suture material with a male clip on one side and a female clip on the other side. The male clip may contain ratchets that, when engaged with the female clip, hold it in place and allow the male clip to adjustably lit within the female clip or even through it to “tighten” the suture when the male and female clips arc engaged. The suture clips are configured to be used in a suture clip applicator that contains paired and opposing needles configured to hold the male clip on one side and the female clip on the other side and to cause the engagement of male and female clips and form a continuous loop through the suture material to enable the closure of fascia or other body tissues.

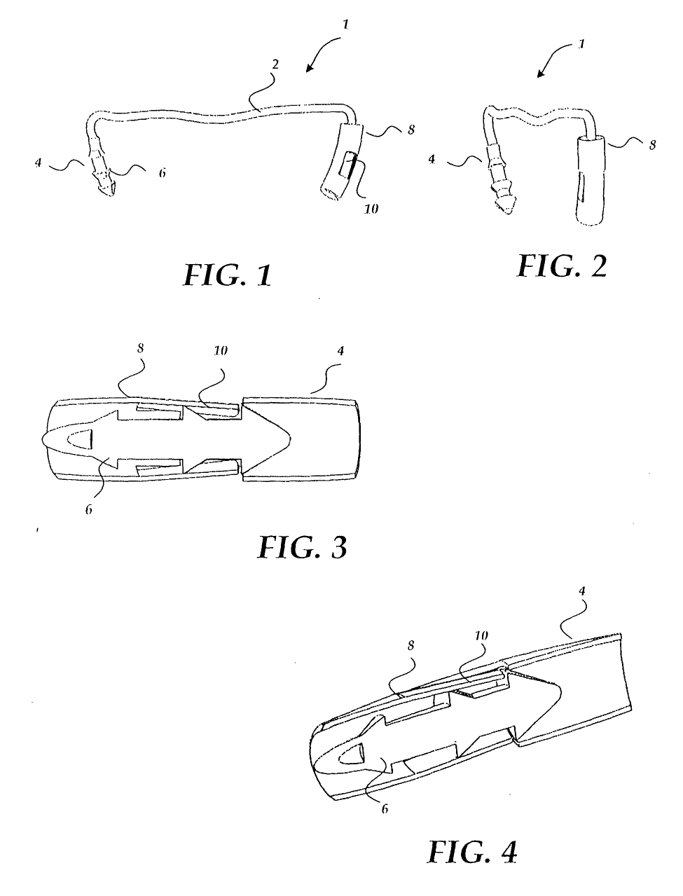

A suture clip is shown in FIGS. 1-4. Suture clip 1 is comprised of suture 2 which may be any type of non-absorbable or absorbable suture material such as polypropylene or braided nylon. In an alternative embodiment, the suture could be an absorbable material such as Vicryl or Monocryl which will dissolve over time. For an external closure, the material may be made of stainless steel. The composition of the suture may also be other suitable types of materials or combinations of materials. On one end of suture 2 is male clip 4. Male clip 4 may be comprised of ratchets 6 and configured to be inserted into female clip 8 located on the other end of suture 2. Female clip 8 may contain engagement recess 10 which interacts with ratchets 6 to hold the male clip within the female clip. Male clip 4 may contain multiple ratchets along its length so that the circumference of suture 2, male clip 4 and female clip 8 when engaged can be decreased with each subsequent ratchet as it engages engagement recess 10. The male clip may pass completely through the female clip to give greater adjustability for the circumference of the complete suture. In an alternative embodiment, the male clip may telescope or fold in on itself within the female suture.

The male clip and the female clip are preferably made of stainless or surgical steel. In an alternate configuration, the clips may be made of other nonabsorbable materials but may also be constructed of dissolvable materials such that when attached to absorbable suture, the entire suture-clip assembly may disappear over time. The main characteristic of the clips are that they have sufficient strength they will not disengage and that the material be tolerated by the body.

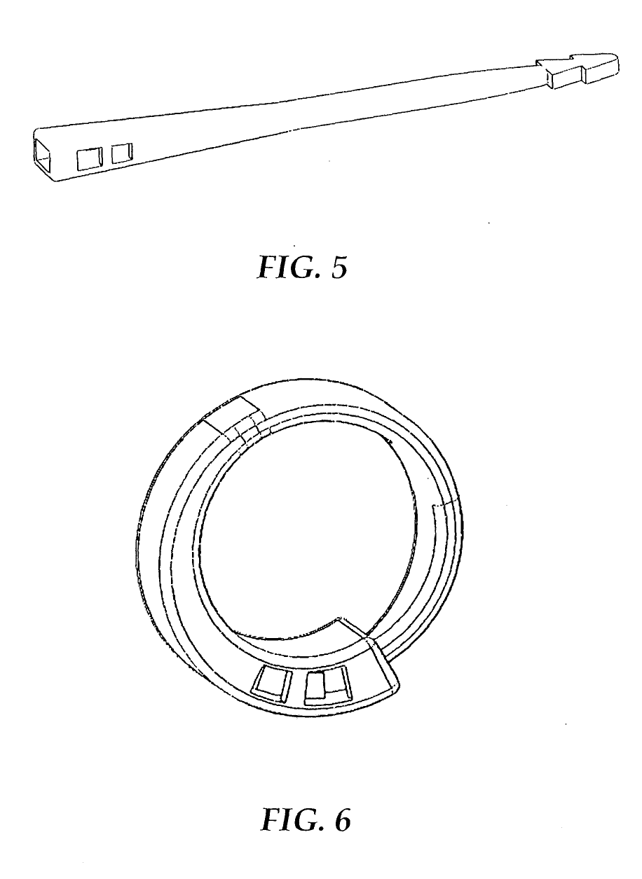

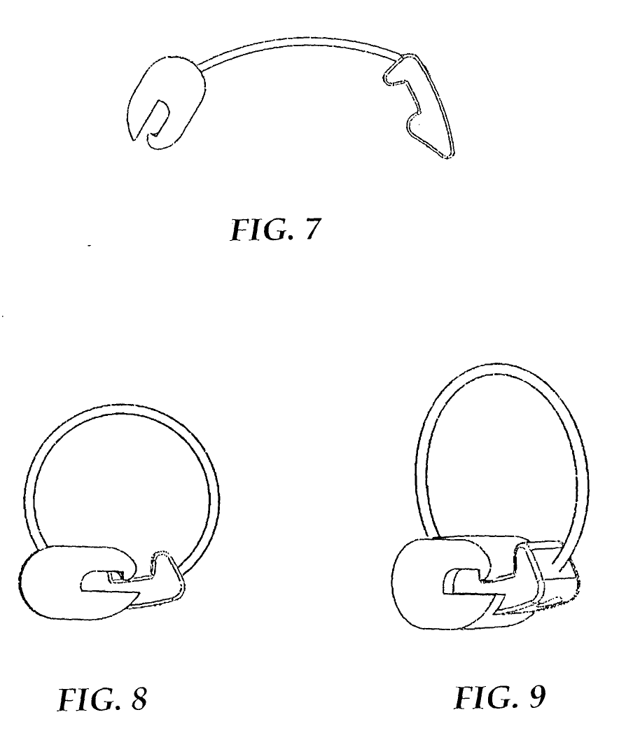

Suture clip 1 may take many other forms such as those shown in FIGS. 5, 6, 7, 8 and 9. The principle feature of the suture clip is that it must have ends that are suitable to be held within a suture clip applicator and capable of being interlocked below the tissue that is being held together. The fastening mechanism may also include low profile attaching methods, where the ends of the suture clip are made of the same material as the suture. As one example, the suture may contain barbs similar to those found in the Quill suture. The barbs may be along the length of the suture and may also be used to engage the female portion of the device to close the suture. A consideration in the configuration of the suture clips and the device is that the suture clips and delivery device should preferably be small enough to minimize tissue puncture.

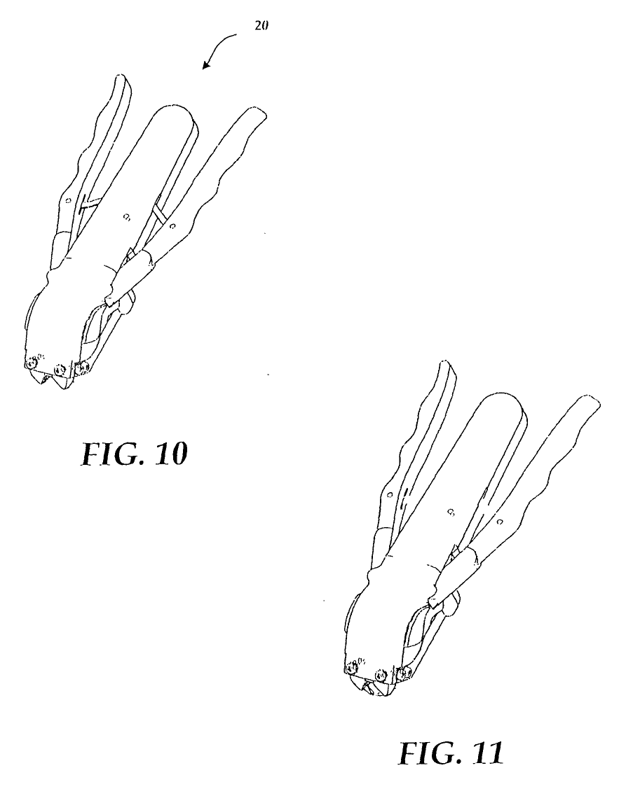

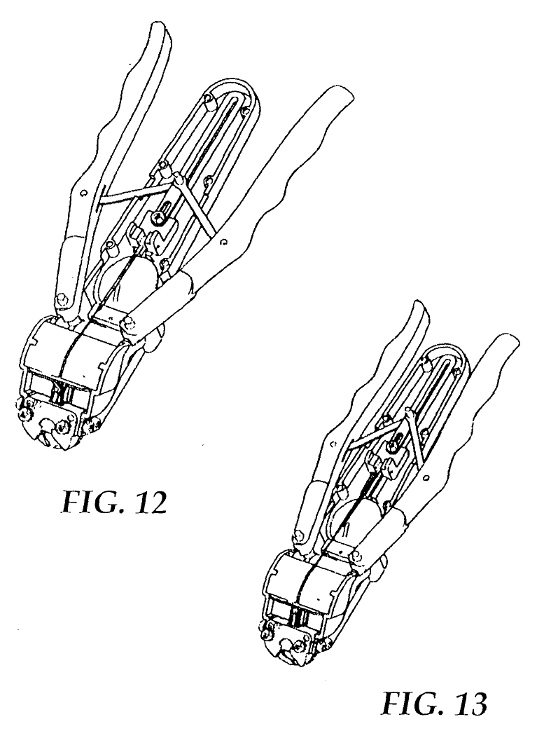

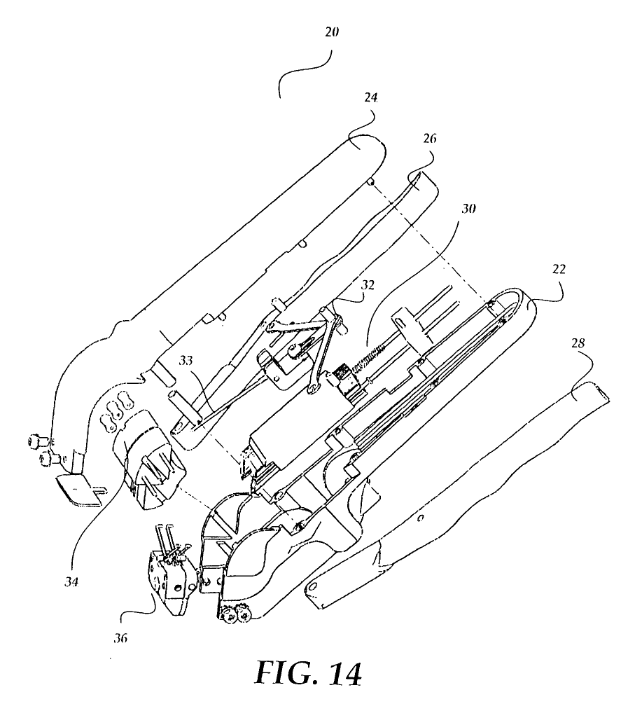

The suture clip applicator that may be used to apply suture clip 1 is shown in FIGS. 10-14. As shown in FIG. 14, suture clip applicator 20 may be comprised base body 22, body cover 24 with first handle 26 and second handle 28 on each side. Within body cover 24 and base body 22 is disposed cartridge 30 for holding suture clips 1, a lever assembly 32, pulley block 34 and needle assembly 36.

In operation, when first handle 26 and second handle 28 are actuated, lever assembly 32 causes wire 33 to pull upwards on a portion of needle assembly 36 through pulley block 34. The action of pulling a portion of needle assembly 36 upwards causes needles within needle assembly 36 to actuate and apply the suture clip as set forth below.

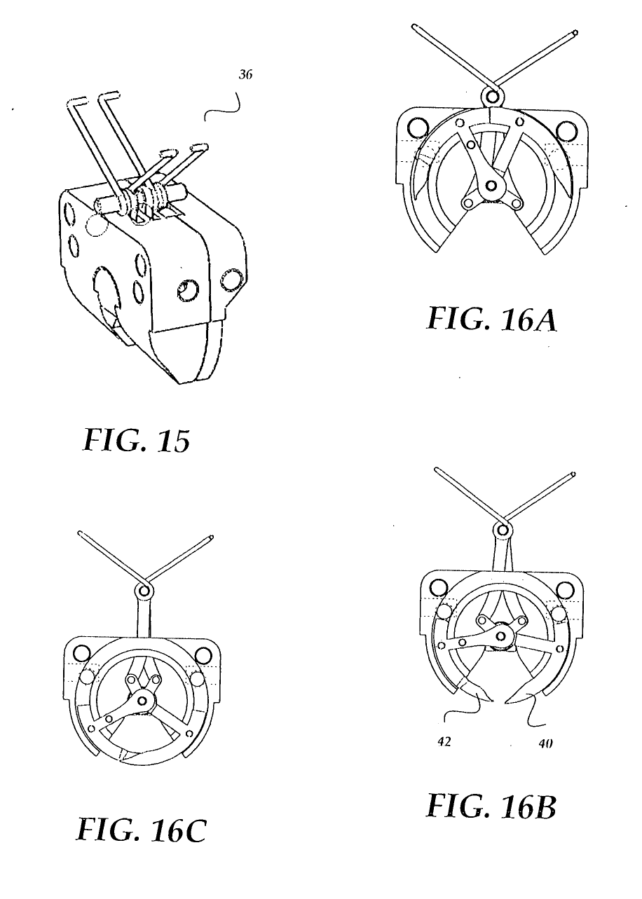

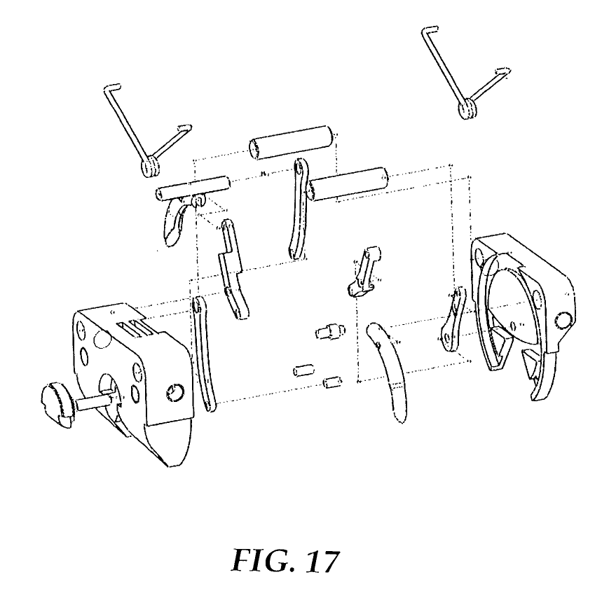

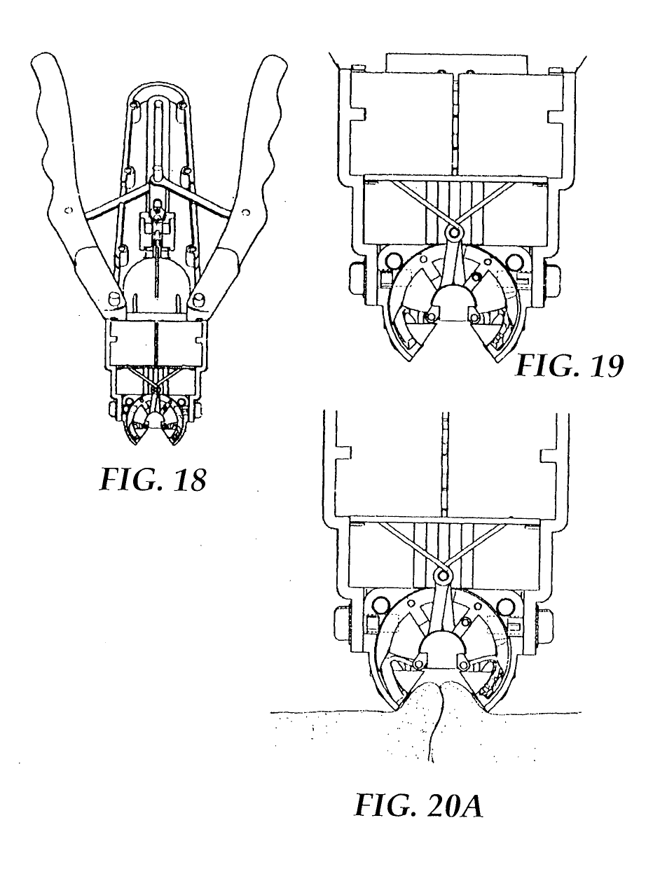

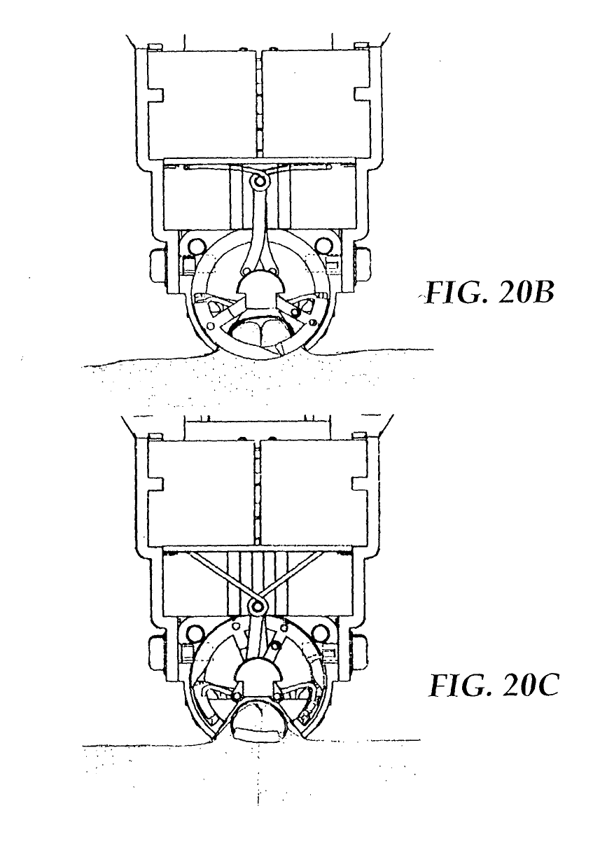

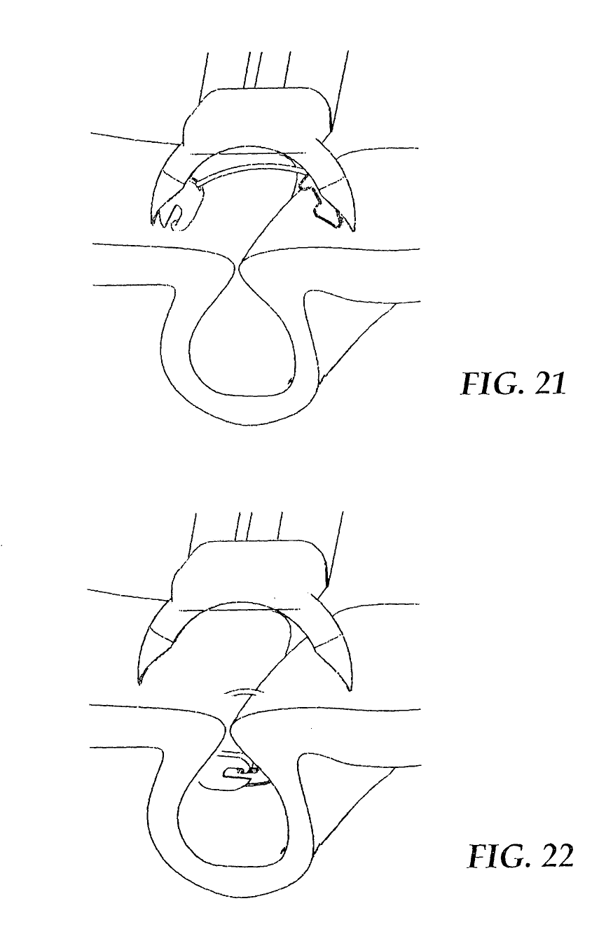

Needle assembly 36 is shown in FIGS. 15, 16 and 17. In this embodiment, needle assembly 36 is configured so that needles 40 and 42 move in a circumferential arc towards one another and overlap. They move from an initial position as shown in FIG. 16A to a partial operated position in FIG. 16B to a fully actuated position in FIG. 16C. Needles 40 and 42 are configured to hold male clip 4 and female clip 8 within an interior channel and to allow suture 2 to extend from the male clip in one of the needles to the female clip in the other needle. In the fully actuated position, male clip 4 and female clip 8 will be engaged so that a complete loop is formed between suture 2, male clip 4 and female clip 8.

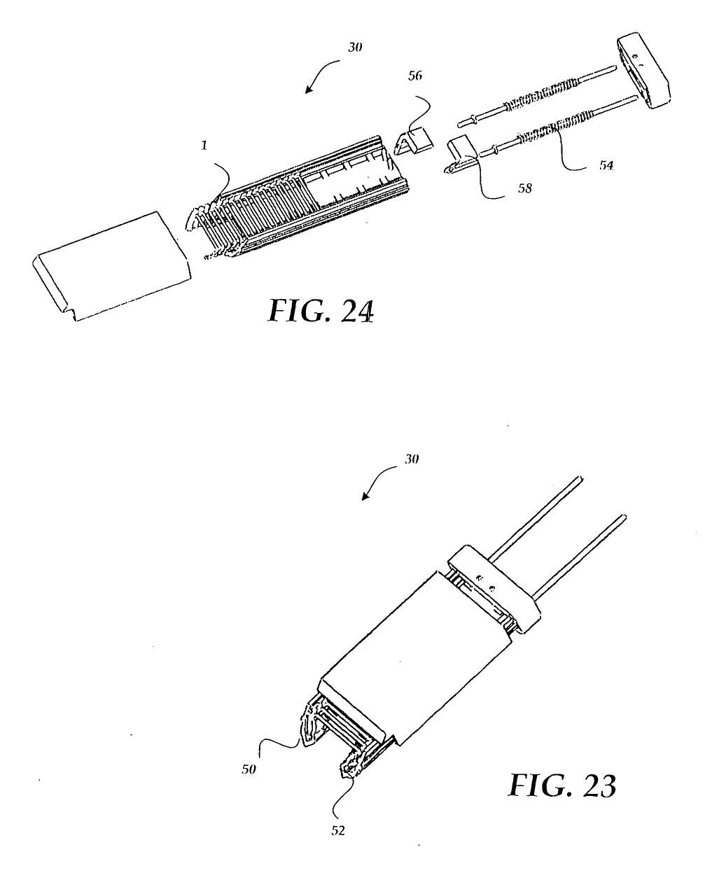

Suture clip applicator 20 is configured to hold and dispense multiple suture clips. This is done through cartridge 30 which is shown in FIGS. 23 and 24. Cartridge 30 contains a plurality of suture clips 1 that are held within female clip holder 50 and male clip holder 52. As shown in FIG. 23, female clip holder 50 and male clip holder 52 may be contoured to fit the dimensions of male clip 8 and female clip 4, respectively. The suture clips are advanced towards the distal end of cartridge 30 through compression spring 54 that biases pusher clips 56 and 58 which in turn pushes on the stack of suture clips 1.

Male clip 4 is compressible and may contain one, two or more ratchets, which allows choices in how tight to make the suture clip when engaged. One aspect of this invention is that when engaging the suture clip, a satisfactory level of tautness can be obtained by the surgeon when using the device. The suture clip application is angled at 45 degrees but other angles devices may also be used so that a surgeon may see the two curvilinear needles coming out of the device and engaging the tissue so that if he wants to play with the tightness he can partially depress the device and, as the needles come out, he can use the left needle to grab the tissue and then move the stapler over and then engage the matching tissue for suturing. This feature keeps a tactile sense in the use of the device. As the surgeon further squeezes the applicator, the tightening across the fascia, for example, may be made to a level that makes the surgeon feel comfortable. In essence, the surgeon has the ability to determine how tight the suture clip will be because of the multiple ratchets of the male clip.

For the diameter and the arc of the needles, the arc may be approximately around the circumference of between 10 and 20 millimeters. However, the size and shape of the needles will vary depending on the intended use. The device may also be made to be pneumatic, which would permit use with closing the sternum following open heart surgery. The suture clip applicator could be used for both internal non-absorbable sutures and external removable sutures, although this would not be the primary use for it because the locking mechanism of the metal under the skin.

The suture clip applicator may be a single use device. The sterile suture clip applicator may come with 20 or 25 pre-loaded suture clips. In an alternative embodiment, the suture clip applicator may be reusable and have suture clip cartridges that may be replaced when required.

In an alternative embodiment, the device may contain multiple opposing hollow curved needles that deploy in parallel. This would permit insertion of multiple sutures at once. In yet another embodiment, the multiple opposing hollow curved needles may be arranged in such a way as to cross each other and automatically deploy a suture similar to a “figure of eight” suture. Such a configuration would provide the advantage of having a two vector approach to tissue closure. A multiplicity of fine sutures may generate sufficient closing tension to be as good as, if not better than, a traditional figure of eight suture.

While the invention has been particularly shown and described with reference to preferred embodiments thereof, it would be understood by those skilled in the art that various changes in form and details may be made therein without departing from the spirit and scope of the invention.

What is claimed as new and desired to be protected is:

1. A suture clip comprising:
a length of suture having a proximal end and a distal end;
a first connector coupled to the proximal end of the suture;
a second connector coupled to the distal end of the suture; and
the first and second connectors being configured to be interconnected to form a continuous loop.

2. A suture clip applicator comprising:
two suture clip needle guides each having a first sharpened end configured for piercing body tissue and a second end configured to receive a suture clip connector;
the suture clip needle guides being arcuately shaped from the first end to the second end and having a C-shaped radial profile forming a guideway for the non-absorbable suture clip connector; and
the suture clip needle guides being actuatable to move circumferentially towards each other such that the first end of each suture clip needle guide overlaps with the first end of other suture clip needle guide and configured to cause suture clip connectors within each guideway to securely interconnect thereby forming a continuous suture capable of holding body tissue together.
